(19) United States
(12) Patent Application Publication
(10) Pub. No.: US 2008/0286882 A1
(43) Pub. Date: Nov. 20, 2008

(54) Method and Apparatus for Measuring the Concentration of a Ligand Contained in a Sample That Is To Be Tested

(75) Inventors: Ulrich Sieben, Vorstettten (DE); Holger Klapproth, Freiburg (DE)

Correspondence Address: THE WEBB LAW FIRM, P.C., 700 KOPPERS BUILDING, 436 SEVENTH AVENUE, PITTSBURGH, PA, 15219, US

(73) Assignee: MICRONAS HOLDING GMBH, Freiburg i.Br. (DE)

(21) Appl. No.: 11/960,090

(22) Filed: Dec. 19, 2007

(30) Foreign Application Priority Data
Apr. 12, 2007 (EP) 07007510

(51) Int. Cl. G01N 33/543; B01J 19/00

(52) U.S. Cl. 436/518; 422/68.1

(57) ABSTRACT

In a method for measuring the concentration of a ligand contained in a sample that is to be tested, at a plurality of test sites that are located on the surface of at least one substrate, in each case at least one receptor that is suitable upon contact with the ligand for specifically binding to said ligand, is immobilized. The sample is brought into contact for a predetermined time with the test sites in such a way that in the sample differently sized diffusion volumes are adjacent to the receptors from which at least one ligand can diffuse to the respective receptor during the predetermined time. After the predetermined time has passed, for each test site a measured signal for the number or density of the binding events at the test site is acquired.

The invention relates to a method for measuring the concentration of at least one ligand contained in a sample that is to be tested, in which at least one receptor that is suitable, when it contacts the ligand, for binding specifically to said ligand is immobilized in each case at at least two test sites that are located on the surface of at least one substrate. The invention also relates to an apparatus for measuring the concentration of at least one ligand contained in a sample to be tested having at least one inlet opening for the measurement chamber containing the sample, which has an interior cavity having defining walls, and on at least one defining wall there are located at least two test sites at which in each case at least one receptor that is suitable when contacting the ligand to bind specifically to said ligand is immobilized, and having at least one sensor to acquire measured signals for the number or density of the binding events at the individual test sites. The invention further relates to an apparatus for measuring the concentration of at least one ligand contained in a sample to be tested having at least one inlet opening for the measurement chamber containing the sample, which has an interior cavity having defining walls, and on at least one defining wall there is located at least one test site at which in each case at least one receptor that is suitable when contacting the ligand to bind specifically to said ligand is immobilized, and having at least one sensor to acquire a measured signal for the number or density of the binding events at the individual test site.

A method of this type and an apparatus of this type are disclosed in DE 102 45 435 B4. The apparatus has a measurement chamber that is embodied as a flow cell and that has an interior cavity having an inlet opening and an outlet opening for the sample that is to be tested. The interior cavity is surrounded by defining walls, namely a flat bottom section, a flat top section parallel thereto at a distance from the bottom section, and side defining walls, on which the inlet and outlet openings are provided. On its surface that borders the interior cavity, the bottom section has an optical waveguide layer on which a plurality of test sites that are laterally separated from each other are located and on which receptors are immobilized in the evanescence field of the waveguide. In order to induce the emission of luminescence radiation as a function of the binding of the ligand contained in the sample to the receptors, optical radiation that is generated with the aid of a radiation source is directed into the waveguides. A radiation receiver that is sensitive to the luminescence radiation is provided under the receptors at each test site. Even though this apparatus has proven itself due to its simple and cost-effective design, it has certain disadvantages. For example, the measurement of the concentration of the ligand is limited to a predetermined measurement range, in particular if the concentration is to be measured simultaneously for a number of different ligands at the individual test sites. If the concentration of at least one ligand lies outside the measurement range, the measured values can be restricted for the respective test site.

Therefore, the object is to provide a method and an apparatus of the aforesaid type that permits the concentration of the ligand to the measured with high precision across a wide range of concentrations.

This object is accomplished with respect to the method of the aforesaid type such that the sample is brought into contact with the test sites for a predetermined length of time, the receptors that are immobilized in the sample at the test sites border on variously sized diffusion volumes, from which the ligand, of which at least one must be present, can diffuse to the respective receptor during a predetermined length of time if the ligand is contained in the diffusion volume, and after the predetermined length of time has passed, for each test site a measured signal for the number or the density of the binding events is acquired at the respective test site.

As a consequence of the variously sized and in some cases different dimensions that the diffusion chambers have, different calibration curves or characteristic curves (measured signal as a function of the exposure time for a given ligand concentration) advantageously result when the measurements are performed for the individual test sites. If at the beginning of a test the ligand concentration at each of the individual test sites is the same, in other words if the ligand is homogeneously distributed in the sample, the measured signal from the test site that borders on a large diffusion volume will reach its limit faster than the measured signal from a test site that borders on a small diffusion volume, because more ligands can diffuse per time unit to the receptors at the respective test site from the larger test volume than can diffuse from the small diffusion volume. This ensures at least one measured signal will always be in a favorable output range across a broad range of concentrations. The receptors, of which at least one must be present, may comprise a nucleic acid or a derivative thereof (DNA, RNA, PNA, LNA, oligonucleotides, plasmids, chromosomes), a peptide, a protein (enzyme, protein, oligopeptides, cellular receptor protein and the complexes thereof, peptide hormone, antibody, and the fragments thereof), a carbohydrate and its derivatives, in particular a glycosylated protein and a glycoside, a fat, a fatty acid, and/or a lipid.

In a preferred embodiment of the invention the measured values are each compared with a predetermined reference value or reference range, and the concentration of the ligand in the sample is determined with the aid of the measured signal that has the least deviation from the reference value or the reference value range or that coincides with the reference value range. By comparing the individual measurement results with the reference value or reference range, a measured signal that is located in a favorable output range, and therefore allows the ligand concentration to be determined precisely after the measurement or exposure time has passed, can be selected. Here, it is even possible to determine the concentration with the aid of a plurality of measured signals that lie within the reference range when the measurement time or exposure time has passed, for example by generating a mean value.

It is advantageous for the reference value to lie between 25% and 75%, more preferably between 40% and 60% of the measured signal that is measured at one test site if essentially all of the receptors of the test site are bound to one ligand, and if the reference value preferably corresponds to the inflection point of a sigmoidal calibration curve that assigns each of the various values for the ligand concentration to respective measured values for the test site. The method then permits an even more precise measurement of concentration, in particular if during the measurement measured values for a correspondingly large number of tests sites are measured with different diffusion chambers.

In a preferred embodiment of the method, the variously sized diffusion volumes are formed by at least one defining wall that borders on the sample at a distance from the test site. The diffusion volume is then defined by the geometry of the measurement chamber and is therefore easy to reproduce.

In a preferred embodiment of the invention, the variously sized diffusion volumes are formed by locating the sample at the individual test sites with different fill levels. This may be accomplished, for example, by providing the test sites at the bottom of a trough or a measurement chamber and disposing the bottom at an incline relative to a horizontal plane or in the shape of steps so that, beginning at one side of the bottom, the sample fill height increases or decreases relative to the diametrically opposite other side of the bottom, for example in the shape of a wedge or in the shape of steps.

With regard to the apparatus of the aforesaid type, the aforesaid object of the invention is accomplished by having the distance between the test site and the wall area of the measurement chamber that is opposite said test site in the direction of a line running normal to the plane of extension of the test site be different at the individual test sites.

When the measurement chamber is filled with the sample, variously sized diffusion volumes result in the sample at the individual test sites, from which, during a predetermined measurement time, ligands can defuse to the receptors located at the test site in order to specifically bind to said receptors. In this way, the measured signals have variously controlled outputs at the individual test sites after the measurement time has passed. This permits at least one measured signal to be located in a favorable output range, in which the concentration of the ligand can be determined with high precision, across a wide range of concentrations.

It is advantageous if the sensor, of which at least one must be present, communicates with an evaluation device, if the evaluation device has a reference value generator for providing at least one reference value or reference range for the measured signals from the test sites, if the evaluation device has at least one comparison device for comparing the measured signals from the individual test sites with a reference value or a reference value range, and if the comparison device is connected to a switching device such that the measured signal that has the smallest deviation from the reference value or from the reference value range, or that coincides with said range, can be applied to a measured signal output of the switching device. With the aid of the evaluation device, a favorably output measured signal for measuring the respective ligand concentration can be selected by means of a comparison with the reference value or the reference value range, and it can be applied to the measured signal output. With the aid of this measured value and the characteristic values assigned to the corresponding test site, for example with the aid of a calibration curve, the concentration of the ligand in the sample can then be determined with a high degree of precision. The evaluation device preferably has a microcomputer. The measured signal output can be located in the microcomputer, in particular in the form of an output for a serial or parallel digital signal.

In a preferred embodiment of the invention, in the case with least two test sites, the distance between the respective test site and the opposing wall area in the direction of the line running normal to the plane of extension of the test site is moveable. By moving the wall areas correspondingly, a liquid that is located in the measurement chamber can be moved and, in particular, transported in a given direction.

With regard to the apparatus of the aforesaid type, the above object is accomplished by having at least one defining wall be moveable relative to at least one additional defining wall in such a way that the diffusion volume from which at least one ligand can diffuse to the receptor during a predetermined measurement time when the interior cavity is filled with sample changes, means to acquire measured signals with different diffusion volumes are provided, the evaluation device has a reference value generator for providing at least one reference value or reference value range for the measured signals from the test site of which at least one must be present, the evaluation device has at least one comparison device for comparing the measured signals acquired with the individual diffusion volumes with the reference value or reference range, and the comparison device is connected to a switching device in such a way that the measured signal that has the smallest deviation from the reference value or reference value range or that coincides with said reference value range, can be applied to a measured signal output of the switching device.

The individual measured signals may also be measured after one another in time, whereby with the individual measurements the defining walls are positioned at various distances relative to reach other in order to form variously large diffusion volumes at the test site. The resulting measured signals may then be temporarily stored in order, with the aid of the evaluation device, to select a measured signal whose output is favorably controlled. The concentration of the ligand in the sample can then be determined with this measured signal and at least one characteristic value for the diffusion volume at which the measured signal was acquired.

A particularly precise measurement of the concentration of the ligand is thereby possible when the reference value corresponds to the inflection point of a sigmoidal calibration curve and the various values for the ligand concentration are each assigned to a measured value for the test site.

It is advantageous if at least one test site at a first chamber wall and the wall area that is opposite this test site be assigned to a second chamber wall, and if said chamber walls are sloped at an angle relative to each other. In this case it is even possible for the chamber walls to be sloped relative to each other in orientations that are perpendicular to reach other. In this way the apparatus can be manufactured in a cost-effective manner with a multitude of different diffusion volumes.

The slope angle can be at least 2.5°, in particular at least 5°, and preferably at least 10°. In this case a slope angle is understood to mean the angle at which the main planes of extension of the opposing chamber walls, which preferably are flat, are sloped relative to reach other. The tests sites preferably are disposed at at least one of the two chamber walls that are sloped relative to reach other.

In another preferred embodiment of the invention, a step or shoulder is formed between at least two wall areas and at least one test site is assigned to each of these wall areas. A measurement chamber having such a stepped inner wall can be manufactured in standard production volumes with high precision using semiconductor technology manufacturing methods.

In some cases it may even be possible for the first chamber wall and/or the second chamber wall facing this first chamber wall to have a plurality of wall areas adjacent to each other and in orientations that are perpendicular to each another, between which steps or shoulders are formed. The apparatus then has compact dimensions and permits a multitude of test sites that border on differing diffusion volumes.

Compact dimensions of the measurement chamber are also made possible when the wall areas between which the steps or shoulders are formed are designed as polygons and preferably are arranged in a checkerboard or honeycomb fashion. The measurement chamber can then be manufactured in a cost-effective manner using standard semiconductor manufacturing processes.

It is advantageous if at least one first test site has a first distance to the opposite wall area in the direction of the line running normal to the plane defined by said test site and at least one second test site has a second distance to the opposite wall area in the direction of the line running normal to the plane defined by said second test site, and if the first distance is at least 1.5 times greater than, in particular at least two times greater than, and preferably at least four times greater than the second distance. These dimensions permit the apparatus to have high measurement dynamics.

It is advantageous if at least one sensor for detecting the ligand-receptor complexes is located in the wall of the measurement chamber on the individual test sites, and the receptor, of which at least one must be present, is immobilized on the sensor. The ligand-receptor complexes may then be detected directly at the given location and therefore have a correspondingly high detection sensitivity.

At least one sensor may be an ion-selective field-effect transistor. If this is the ease, the apparatus can be manufactured cost-effectively.

In a preferred embodiment of the invention, at least one sensor is an optical radiation sensor that is sensitive to the luminescence radiation that can be excited depending on the binding of the ligand to the receptor. The luminescence radiation may be excited by an excitation radiation that can be generated by means of a radiation source. The radiation source may be embodied as a semiconductor element integrated into the wall of the measurement chamber. However, the luminescence radiation may also be excited by chemical means. In this way it is possible to eliminate one radiation source.

Examples of embodiments of the invention are explained in greater detail below based on the drawing. The drawing shows:

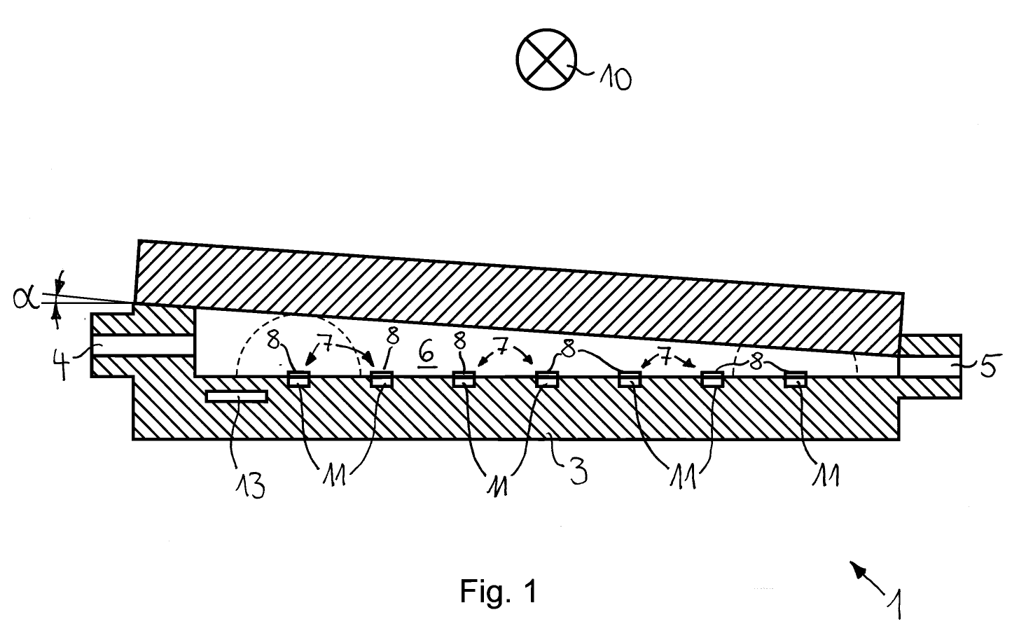

FIG. 1 a cross section through a first example of an embodiment of an apparatus for measuring the concentration of a ligand contained in a sample that is to be tested,

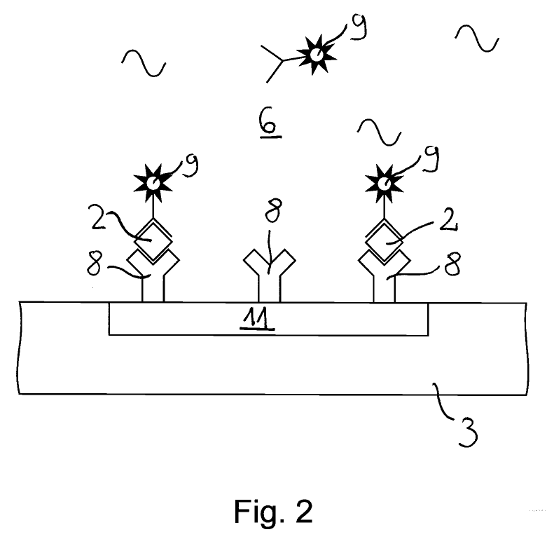

FIG. 2 an enlarged partial view of FIG. 1, that shows a test site on which receptors are immobilized on an optical sensor,

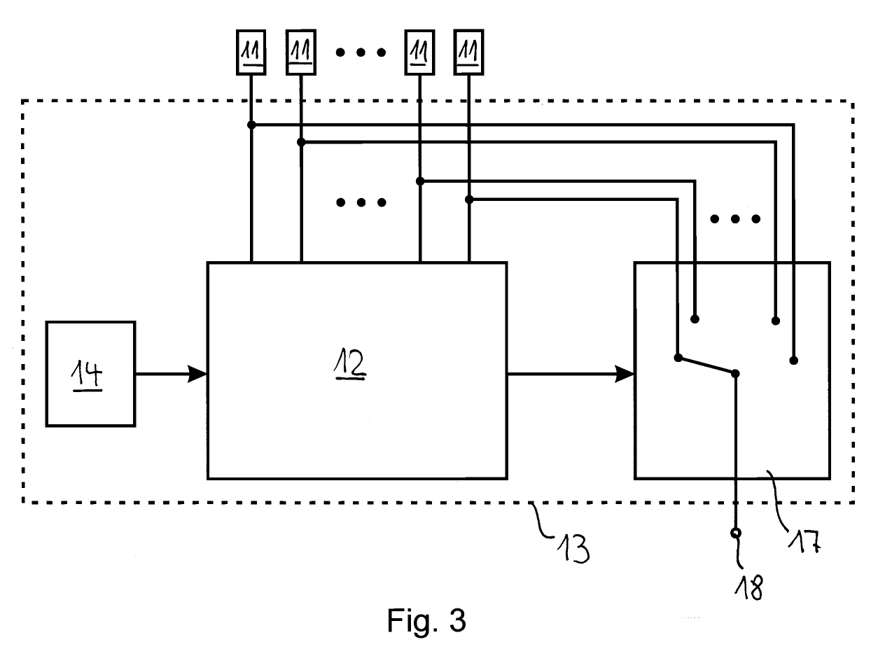

FIG. 3 a schematic diagram of an evaluation device of the apparatus,

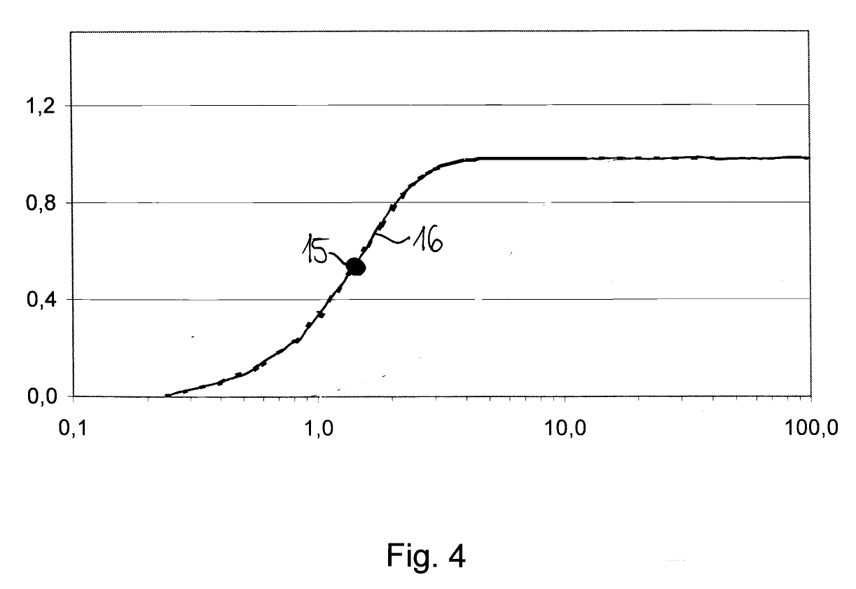

FIG. 4 a graphical representation of a calibration curve for a test field of the apparatus, in which time is plotted on the abscissa and the amplitude of the measurement signal for the test site is plotted on the ordinate,

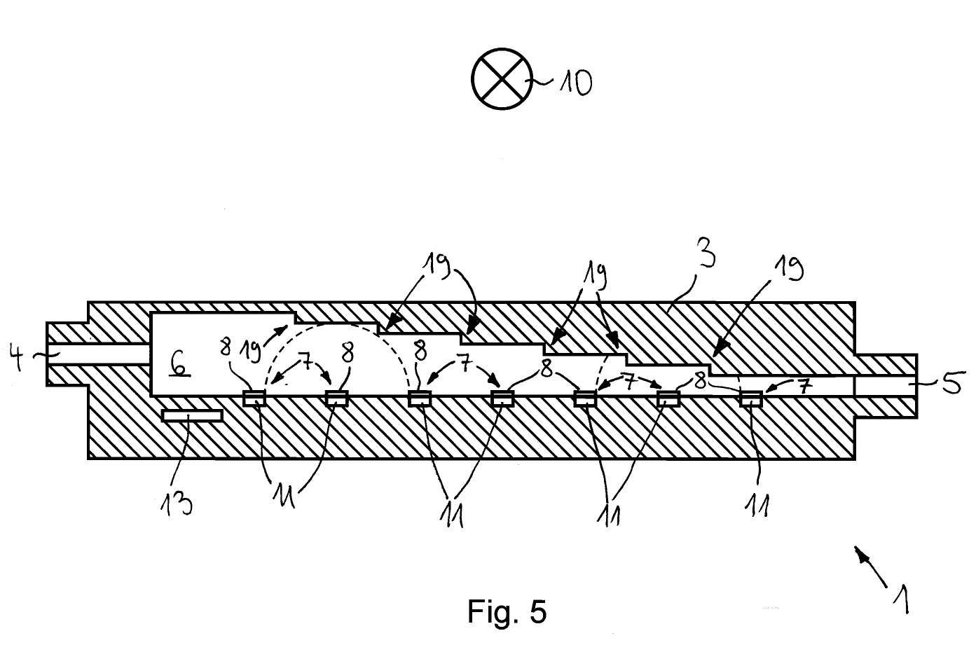

FIG. 5 a cross section through a second example of an embodiment of the apparatus,

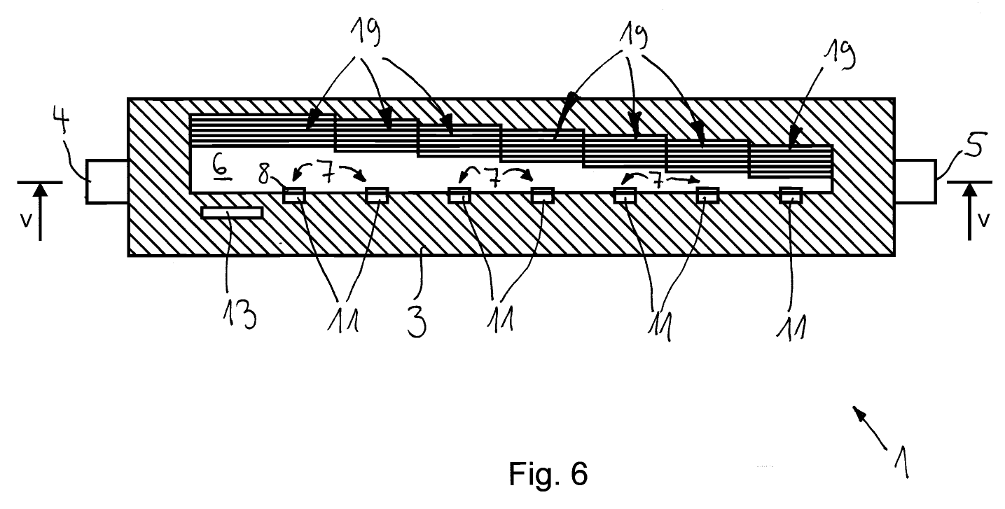

FIG. 6 a cross section through a third example of an embodiment of the apparatus,

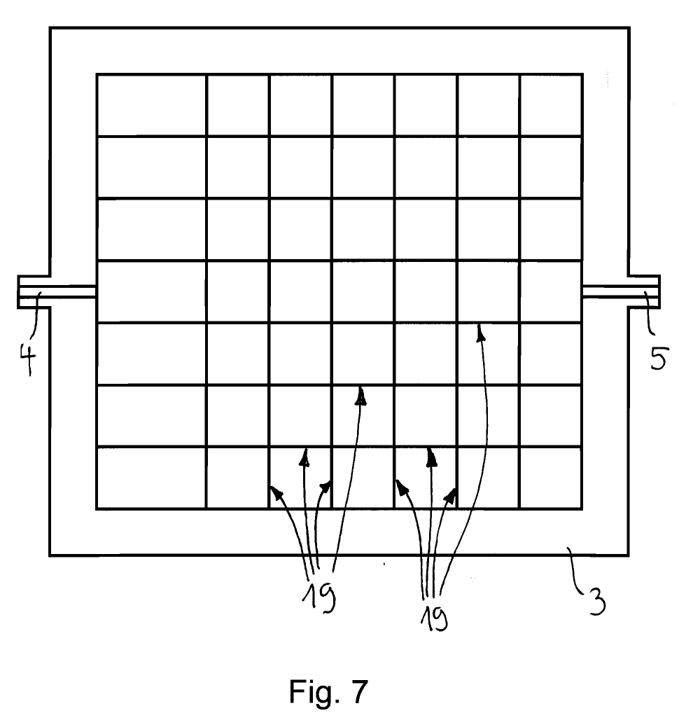

FIG. 7 a longitudinal sectional view through the third example of an embodiment,

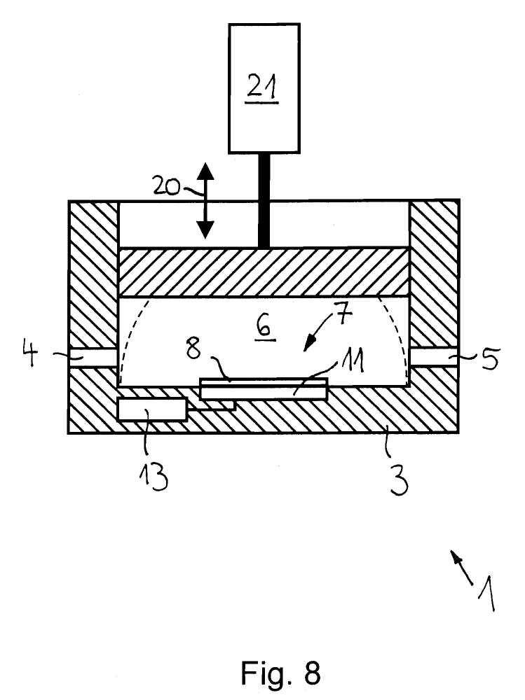

FIG. 8 a cross section through a fourth example of an embodiment of the apparatus in which the defining walls of the measurement chamber are adjustable relative to each other, and

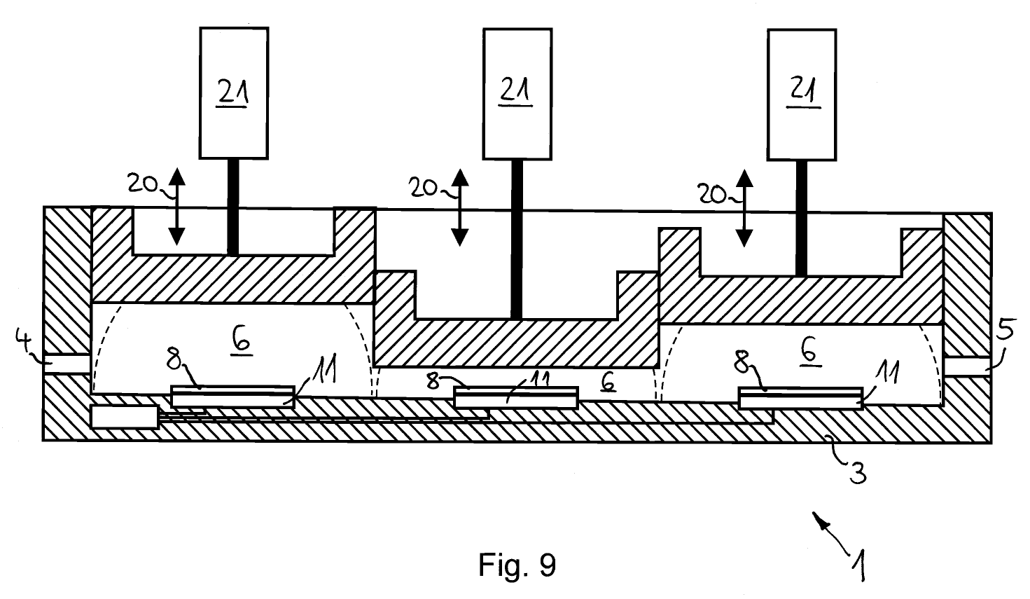

FIG. 9 an apparatus that has a plurality of defining walls disposed adjacent to reach other and that can be moved relative to an additional defining wall.

An apparatus that is shown in complete form in FIG. 1 and is identified by 1 for measuring the concentrations of a plurality of ligands of various ligand types contained in a sample that is to be tested has a measurement chamber 3 having an interior cavity 6 that is closed off with the exception of an inlet opening 4 and an outlet opening 5. The interior cavity 6 is defined in the upward, downward, and lateral directions by defining walls.

On a defining wall that forms the bottom of the measurement chamber 3 there are disposed a plurality of test sites 7 that are spaced apart from each other and that are arranged adjacent to each other in rows and columns in a matrix-like shape. A receptor 8 that is binding-specific for a ligand 2 of a particular ligand type is immobilized on each of the test sites 7 of the individual rows. The receptors of a first row of test sites differ from the receptors of a second row of test sites. Of course it is also possible for the receptors 8 of a plurality of rows having test sites 7, or all of the test sites 7, to belong to the same receptor type.

If receptors 8 for at least two receptor types are immobilized in the first measurement chamber 3, they do not necessarily have to be disposed in the same row of the test site matrix. Instead, receptors for various receptor types may be disposed in a first test site row, although only receptors of the same receptor type are immobilized at the same test site 7. Therefore, only ligands of a predetermined ligand type can bind to a particular test site 7.

At the individual test sites 7 the distance between the test site 7 and an opposing wall area of the measurement chamber in the direction of the line running normal to the plane of extension of the test site 7 varies. If the internal cavity 6 is filled with the sample, variously sized diffusion volumes, which are present in the sample and from which at least one ligand can diffuse during a predetermined exposure time to receptors 8 located at the respective test site 7, are adjacent to the test site.

A diffusion volume is understood to mean the partial volume of the interior cavity 6 of the measurement chamber 3 lying within a sphere whose center point is located at the test site and whose radius corresponds to the mean rate of diffusion of the ligand in the sample multiplied by the exposure time. The exposure time is understood to mean the time during which the sample is in contact with the test site 7 until the measurement is finished. In FIG. 1 the diffusion volume for two of the test sites 7 is indicated by dashed lines. It can be clearly seen that a larger diffusion volume is allocated to the test site 7 on the left side of the drawing than to the test site 7 located on the right side.

In FIG. 2 it can be seen that the ligands 2 are marked with an optical marker 9 by means of a detection antibody. Preferably the sample is first brought into contact with the receptors 8 for a predetermined exposure time, and then components of the sample that are not bound to a receptor 8 are removed from the measurement chamber 3, for example by directing a rinsing liquid through the measurement chamber 3. Then the ligands 2 that are bound to a receptor 8 are marked with the marker 9 by filling the interior cavity 6 of the measurement chamber 3 with a liquid that contains the marker 8. After the markers 9 have bound to the ligands 2, free markers 9 are removed from the measurement chamber 3, for example by rinsing out the interior chamber 6 again.

However, it is also possible to mark the ligands 2 with the marker 9 before the sample is brought into contact with the test sites 7. In this case the sample containing the marked ligands 2 is first brought into contact with the test sites 7 for a predetermined exposure time, and then components of the sample that are not bound to a receptor 8 are removed from the measurement chamber 3.

After the ligands 2 are bound to the receptors 8 and marked, the test sites are irradiated by means of a radiation source 10 with an optical excitation radiation, which excites the markers 9 to emit luminescence radiation. The wavelength of the luminescence radiation differs from the wavelength of the excitation radiation.

The luminescence radiation is detected with the aid of optical sensors 11 that are integrated into the wall of the measurement chamber 3 at the individual test sites 7, in each case directly beneath the receptors 8. An optical blocking filter for the excitation radiation (not specifically shown in the drawing), which allows the luminescence radiation to pass, is provided between the receptors 8 and in the sensors 11. With the aid of the sensors 11, a measured signal, whose amplitude is dependent on the number of markers at the respective test site 7, is generated for each test site 7.

As can be seen in FIG. 3, each of the sensors 11 has a measured signal output that is connected to an input of the comparison device 12 of an evaluation device 13. A further input to the comparison device 12 is connected to a reference value generator 14, which provides a reference value. The reference value corresponds to the inflection point 16 of a sigmoidal calibration curve 17 that assigns various values for the ligand concentration to each measured value for the respective test site 7 (FIG. 4). With the aid of the comparison device 12, the measured signals from the individual sensors 11 are each compared with the reference value. The comparison device 12 is connected under control to a switching device 17 in such a way that the measured signal that has the lowest deviation from the reference value, or that coincides with the reference value, is supplied to a measured signal output of the switching device. The concentration of the ligand 2 in the sample is determined with the aid of this measured signal and a calibration curve. Characteristic values for the diffusion volume at the test site 7 that is assigned to the measured signal and the binding constant of the receptors 8 located at the test site 7 are taken into account in the calibration curve. The calibration curve can be generated by means of measurement with the aid of at least one test and/or computationally, for example with the aid of a suitable model.

The evaluation device 13 is only shown schematically in FIG. 3. Specifically, it may have a microcomputer in which the individual measured signals are compared with the reference value by means of an operating program that is run in the microcomputer. The reference value generator may have a data memory in which the reference value is preferably stored in digital form. The individual measured signals may be compared with the reference value sequentially or simultaneously. It is also conceivable that the reference value generator 14 provides a dedicated reference value for each test site 7 and/or for each receptor type.

In the example of the embodiment shown in FIG. 1 the inner surfaces of the measurement chamber defining wall that are provided at the test sites 7 and that face the interior cavity 6, and the defining wall that is opposite them are sloped relative to each other at a given angle α. The inner surfaces of both defining walls are flat, so that the interior cavity 6 has an approximately wedge-shaped cross section.

In the examples of embodiments shown in FIGS. 5 to 7, the defining wall opposite the test sites 7, namely the cover portion of the measurement chamber 3, has steps 19 on the wall's inner surface facing the interior cavity 6. Each step 19 is opposite and roughly centered on a test site 7. The wall area of step 19 that faces the test site 7 extends roughly parallel to the inner surface of the opposite defining wall, which contains the test site 7.

In the example of the embodiment shown in FIGS. 6 and 7, the defining wall of the interior cavity 6 that faces the test sites 7 is stepped in directions that extend at right angles to each other. In the view of the defining wall, the individual wall areas of the measurement chamber 3 that faced the test sites 7 and that are formed by the steps are disposed adjacent to each other in the form of a matrix in a plurality of rows and columns. This results in a roughly checkerboard pattern of steps.

The sensors 11 for detecting the binding events are configured as ion-selective field-effect transistors that are integrated into the wall of the measurement chamber 3 directly beneath the receptors 8. The evaluation device 13 is also integrated into the wall of the measurement chamber 3.

In the example of the embodiment shown in FIG. 8, a defining wall of the measurement chamber 3 can be moved in the manner of a piston relative to the opposite defining wall containing the test site 7 toward and away from the measuring site 7 in the direction of the double-ended arrow. This makes it possible to set different diffusion volumes from which the ligand 2, of which at least one must be present, can defuse during a predetermined measurement time to the receptor 8. In FIG. 8 a possible diffusion volume is outlined by a dashed line. The movement of the defining wall is accomplished with the aid of an actuator 21.

In order to measure the concentration of the ligand 2 a first test is performed in which the sample is transferred into the interior cavity 6 in such a way that the ligand 2, of which at least one must be present, that is marked with the marker 9 can bind at the receptor 8. Then components of the sample that are not bound to the receptor 8 are removed from the interior cavity 6, and a first measurement signal for the number of binding events at the test site 7 is acquired with the aid of the sensor 11. The measured signal is saved. Then any binding sites between a ligand and the receptor, of which at least one must be present, that may be present are broken, for example by applying heat, and the separated ligands are removed from the measurement chamber 3. In order to break the ligand-receptor bonds, the measurement chamber 3 may have a heater.

For a second test the distance between the defining walls is adjusted with the aid of an actuator 21 in order to change the diffusion volume. The second test is then performed in a corresponding manner. Then, if needed, at least one further test is performed, with the diffusion volume being changed in each test. The resulting measured values are compared with the reference value. With the aid of the measured signal that has the smallest deviation from the reference value, or that coincides with the reference values and with the aid of a calibration curve, the concentration of the ligand 2 in the sample is determined.

In FIG. 9 it can be seen that the apparatus may also have a plurality of adjacent defining walls that are movable relative to an additional opposing defining wall in the direction indicated by the double-ended arrow 20. With the aid of this movable defining wall disposed in the shape of a matrix, it is possible to transport a liquid present in the measured chamber 6, for example from the inlet opening 4 to the outlet opening 5.

1. A method for measuring the concentration of at least one ligand contained in a sample that is to be tested, and at at least two test sites that are disposed on the surface of a least one substrate, in each case at least one receptor that is suitable upon contact with the ligand for specifically binding to said ligand is immobilized, and the sample is brought into contact with the test sites for a predetermined time in such a way that in the sample differently sized diffusion volumes border on receptors that are immobilized at the test sites and from said volumes at least one ligand can diffuse to the respective receptor during a predetermined time if the ligand is contained in the diffusion volume, and after the predetermined time for each test site in each case one measured signal for the number or density of the binding events at the test site is acquired.

2. The method of claim 1, wherein the measured values are each compared with a predetermined reference value or reference range, and the concentration of the ligand in the sample is determined with the aid of the measured signal that has the lowest deviation from the reference value or the reference value range, or that it coincides with same.

3. The method of claim 1, wherein the reference value range lies between 25% and 75%, preferably between 40% and 60%, of the measured signal that is measured at a test site if essentially all of the receptors of the test site are bound to a ligand and the reference value preferably corresponds to the inflection point of a sigmoidal calibration curve that assigns each of the various values for the ligand concentration to a respective measured value for the test site.

4. The method of claim 1, wherein the different diffusion volumes are formed by at least one defining wall that borders on the sample at a distance from the test site.

5. The method of claim 1, where in the different diffusion volumes are formed by disposing the sample with a different fill height at the individual test sites.

6. An apparatus to measure the concentration of at least one ligand contained in a sample to be tested having at least one inlet opening for the measurement chamber containing the sample, which has an interior cavity with defining walls, and at at least one defining wall at least two test sites are disposed on which at least one receptor that, upon contact with a ligand, is suitable for specifically binding to said ligand is immobilized, and having at least one sensor for acquiring measured signals for the number or density of the binding events at the individual test sites, wherein in the individual test sites the distance between the test site and an area of the wall of the measurement chamber that faces the test sites in the direction of a line running normal to the plane of extension of the test site has various sizes.

7. The apparatus of claim 6, wherein at least one sensor communicates with an evaluation device, the evaluation device has a reference value generator for providing at least one reference value or reference range for the measured signals from the test sites, the evaluation device has at least one comparison device for comparing the measured signals of the individual test sites with the reference value or the reference value range, and the comparison device is connected to a switching device in such a way that the measured signal that has the smallest deviation from the reference value or reference value range, or that coincides with the reference value range, can be applied to a measured signal output of the circuit device.

8. The apparatus of claim 6, wherein in at least two test sites the distance between the respective test site and the wall area that is opposite to the test site in the direction of a line running normal to the plane of extension of the test site is moveable.

9. A device to measure the concentration of a least one ligand contained in a sample to be tested having at least one inlet opening for the measurement chamber containing the sample, which has an interior cavity with defining walls, and at least one test site at which at least one receptor which upon contact with the ligand is suitable for specifically binding to said ligand is immobilized at at least one defining wall, and having at least one sensor for acquiring a measured signal for the number or density of the binding events at the test site, wherein at least one defining wall is movable relative to at least one additional defining wall such that the diffusion volume from which the ligand, of which at least one must be present, can, when the internal cavity is filled with sample, defuse to the receptor during a predetermined measurement time, and means are provided to acquire measured signals with different diffusion volumes, and the evaluation device has a reference value generator for providing at least one reference value or reference range for the measured signals of the test site, of which at least one must be present, and the evaluation device has at least one comparison device to compare the measured signals acquired for the individual diffusion volumes with the reference value or the reference value range, and the comparison device is connected to a switching device in such a way that the measured signal that has the least deviation from the reference value or the reference value range, or that coincides with the reference value range, can be applied to a measured signal output of the switching device.

10. The apparatus of claim 9, wherein the reference value corresponds to the inflection point of a sigmoidal calibration curve, and the various values for the ligand concentration are each assigned to a measured value for the test site(s).

11. The apparatus of claim 9, wherein at least one test site is disposed on a first chamber wall, and the wall area that is located opposite this test site is disposed on a second chamber wall, and these chamber walls are sloped at an angle (α) relative to each other.

12. The apparatus of claim 9, wherein the angle (α) is it least 2.5°, in particular at least 5°, and preferably at least 10°.

13. The apparatus of claim 9, wherein a step or a shoulder is formed between at least two wall areas, and at least one test site is assigned to each of these wall areas.

14. The apparatus of claim 9, wherein the first chamber wall and/or a second chamber wall opposite said first chamber wall each have a number of wall areas disposed adjacent to each other in orientations perpendicular to reach other, between which steps or shoulders are formed.

15. The apparatus of claim 9, wherein the wall areas between which the steps or the shoulders are formed are designed as polygons and preferably are disposed in checkerboard-like or honeycomb-like manners.

16. The apparatus of claim 9, wherein at least one first test site has a first distance to the opposing wall area in the direction of the line running normal to the plane of extension of said test site, and at least one second test site has a second distance to the opposite wall area in the direction of a line running normal to the plane of extension of said test site, and the first distance is at least 1.5 times the second distance, in particular at least two times greater than the second distance, and preferably at least four times greater than the second distance.

17. The device of claim 9, wherein at least one sensor for detecting the ligand-receptor complexes is located in the wall of the measurement chamber at each of the individual test sites, and at least one of the receptors is immobilized on the sensor.

18. The apparatus of claim 9, wherein at least one sensor is an ion-selective field-effect transistor.

19. The device of claim 9, wherein at least one sensor is an optical radiation sensor that is sensitive to luminescence radiation that is excitable as a function of the binding of the ligand to the receptor.
